SEQUENCE LISTING (13 sequences)

SEQ ID NO: 1 - DNA, 22 bp, Artificial Sequence
  Description of Artificial Sequence Synthetic oligonucleotide
aaaaacaaacaagacccttgag

SEQ ID NO: 2 - DNA, 36 bp, Artificial Sequence
  Description of Artificial Sequence Synthetic probe
gtctgtcgtgcgaactcttctcaagggtcttgtttg

SEQ ID NO: 3 - DNA, 17 bp, Artificial Sequence
  Description of Artificial Sequence Synthetic oligonucleotide
gagttcgcacgacagac

SEQ ID NO: 4 - DNA, 17 bp, Artificial Sequence
  Description of Artificial Sequence Synthetic oligonucleotide
catacccgtaatagcgt

SEQ ID NO: 5 - DNA, 34 bp, Artificial Sequence
  Description of Artificial Sequence Synthetic probe
atgcgaataattggtttacgctattacgggtatg

SEQ ID NO: 6 - DNA, 15 bp, Artificial Sequence
  Description of Artificial Sequence Synthetic oligonucleotide
accaattattcgcat

SEQ ID NO: 7 - DNA, 22 bp, Artificial Sequence
  Description of Artificial Sequence Synthetic oligonucleotide
catacctgtaccgtaatagcgt

SEQ ID NO: 8 - DNA, 39 bp, Artificial Sequence
  Description of Artificial Sequence Synthetic probe
atgcgaataattggtttacgctattacggtacaggtatg

SEQ ID NO: 9 - DNA, 39 bp, Artificial Sequence
  Description of Artificial Sequence Synthetic oligonucleotide
  Description of Combined DNA/RNA Molecule Synthetic oligonucleotide
atgcgaataattggutuacgcuattacggtacaggtatg

SEQ ID NO: 10 - DNA, 21 bp, Artificial Sequence
  Description of Artificial Sequence Synthetic oligonucleotide
ttgcatccagtacttcaatac

SEQ ID NO: 11 - DNA, 34 bp, Artificial Sequence
  Description of Artificial Sequence Synthetic probe
aaaaagacgtagggtattgaagtactggatgcaa

SEQ ID NO: 12 - DNA, 29 bp, Artificial Sequence
  Description of Artificial Sequence Synthetic oligonucleotide
ttgcatccagtacttcaataccctacgtc

SEQ ID NO: 13 - DNA, 38 bp, Artificial Sequence
  Description of Artificial Sequence Synthetic oligonucleotide
tttcgtttgcctgctttatctctgttctactatttccg

(19) United States
(12) Patent Application Publication
(10) Pub. No.: US 2020/0209229 A1
(43) Pub. Date: Jul. 2, 2020

(54) SEQUENTIAL MULTIPLEX WESTERN BLOTTING

(71) Applicant: Bio-Rad Laboratories, Inc., Hercules, CA (US)

(72) Inventors: William STRONG, El Cerrito, CA (US); Eli HEFNER, Fairfield, CA (US)

(21) Appl. No.: 16/722,706

(22) Filed: Dec. 20, 2019

Related U.S. Application Data
(60) Provisional application No. 62/785,389, filed on Dec. 27, 2018.

(51) Int. Cl. G01N 33/543; G01N 33/58; C12Q 1/6869; C12Q 1/6834

(52) U.S. Cl. CPC G01N 33/54306; G01N 33/58; C12Q 2563/179; C12Q 1/6834; C12Q 1/6869

(57) ABSTRACT

Described are methods and compositions for sequential multiplex detection of target analytes in a sample. The method comprises contacting the sample comprising analytes immobilized on a solid support with binding agents that specifically bind an analyte in the sample, wherein each of the binding agents binds a different analyte and is attached to a single-stranded nucleic acid molecule comprising a unique sequence. The sample is then contacted with a labeled complementary nucleic acid molecule that binds the single-stranded nucleic acid molecule attached to one binding agent. The signal from the label is detected, and then reduced or eliminated. The sample can be simultaneously contacted with a second labeled complementary nucleic acid molecule that binds a different binding agent, and the signal from the second label is detected. The process is repeated for each additional analyte in the sample, thereby sequentially detecting the presence of the analytes in the sample.

### CROSS-REFERENCES TO RELATED APPLICATIONS

The present application claims the benefit of priority to U.S. Provisional Patent Application No. 62/785,389, filed Dec. 27, 2018, the contents of which are hereby incorporated by reference herein in their entirety for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 094260-1163920-116210US_SL.TXT, created on Dec. 17, 2019, 3,359 bytes, machine format IBM-PC, MS-Windows operating system, is hereby incorporated by reference in its entirety for all purposes.

### BACKGROUND OF THE INVENTION

Existing methods for multiplex Western blotting have limited multiplex capability because they require primary antibodies of different species, multiple secondary antibodies, and there is spectral overlap of fluorescent dyes used to detect the antibodies. Strip-and-reprobe methodologies have the disadvantage of being long and tedious, requiring re-blocking and additional long antibody incubation steps for detection of each subsequent target, and require the use of harsh reagents (detergents, reducing agents, low pH) and/or heat between cycles, which can strip the target antigen from the surface of the membrane. In addition, the use of multicolor detection requires costly instruments, which can affect adoption of the method.

The instant application describes a solution to the problems of existing assays.

### BRIEF SUMMARY OF THE INVENTION

Described herein are methods and compositions that are useful for sequentially detecting the presence of target analytes in a sample. In some embodiments, the target analytes are immobilized on a solid support.

In one aspect, the method comprises:
i) contacting the sample comprising two or more analytes immobilized on a solid support with two or more binding agents that specifically bind an analyte in the sample, wherein each of the analyte-specific binding agents binds a different analyte and is attached to a single-stranded nucleic acid molecule comprising a unique sequence;
ii) contacting the sample with a nucleic acid molecule comprising a first detectable label and a sequence having a region of complementarity that binds the unique sequence attached to a first analyte-specific binding agent;
iii) detecting a signal from a first detectable label;
iv) reducing the signal of the first detectable label;
v) contacting the sample with a nucleic acid molecule comprising a second detectable label and a sequence having a region of complementarity that binds a unique sequence attached to a second analyte-specific binding agent; and
vi) detecting a signal from a second detectable label,
thereby sequentially detecting the presence of the two or more analytes.

In some embodiments, the method further comprises repeating steps (iv)-(vi) for each additional target analyte immobilized on the solid support. In some embodiments, steps (iv) and (v) occur simultaneously.

In some embodiments, reducing the signal of the detectable label comprises quenching, inactivating, or removing the signal or detectable label. In some embodiments, removing the signal comprises digesting the nucleic acid comprising the detectable label. In some embodiments the digesting involves using a restriction enzyme and/or a DNA glycosylase combined with endonuclease VIII. In some embodiments, removing the signal involves photocleavage of the nucleic acid backbone comprising a photocleavable spacer. In some embodiments, removing the signal comprises displacing the nucleic acid strand comprising the detectable label. In some embodiments, displacing the nucleic acid strand includes using a polymerase enzyme that has a strand displacement function. In some embodiments, displacing the nucleic acid strand comprises using toehold exchange strand displacement such as described in Yurke, B et al, 2000, Nature 406, p 605-608 and Zhang, D Y and Winfree, E, 2009, J Am Chem Soc 131, p 17303-17314. In some embodiments, reducing the signal of the detectable label does not remove target analytes from the solid support. In some embodiments, the first and second detectable label(s) is/are the same or different.

In some embodiments, the nucleic acid molecule comprising the detectable label forms a duplex along at least a portion of the unique sequence.

In some embodiments, the single-stranded nucleic acid molecule is attached to the binding agent via a 5′ phosphate group, an amine group, carboxyl group, hydroxyl group, a sulfhydryl group, click chemistry, copper(I)-catalyzed azide-alkyne cycloaddition (CuAAC), strain-promoted azide-alkyne cycloaddition (SPAAC), strain-promoted alkyne-nitrone cycloaddition (SPANC), or a linker. In some embodiments, the single-stranded nucleic acid molecule is attached via reductive amination following oxidation of carbohydrates on the binding agent. In some embodiments, a linker comprising biotin, streptavidin, protein A, protein G, protein A/G, or protein L is used to attach the single-stranded nucleic acid.

In some embodiments, the binding agent comprises an antibody or antigen-binding fragment thereof, a nanobody, affibody or other antibody mimetic, an aptamer, a receptor, a ligand, a peptide, a lectin, a nucleic acid molecule, or a small molecule.

In another aspect, a method for sequentially detecting the presence of two or more target analytes in a sample is described. In some embodiments, the target analytes are immobilized on a solid support. In some embodiments the target analytes are associated with the solid support through a binding agent that specifically binds the target analyte, such as an antibody, in a sandwich-type assay modality.

In some embodiments, the method comprises:
i) contacting a solid support comprising at least two different target analytes immobilized thereon with at least a first binding agent that specifically binds a first target analyte in the sample and at least a second binding agent that specifically binds a second target analyte in the sample, wherein the first analyte-specific binding agent is attached to a first nucleic acid molecule comprising a unique sequence, and the second analyte-specific binding agent is attached to a second nucleic acid molecule comprising a unique sequence;
ii) contacting the first nucleic acid molecule with a nucleic acid molecule comprising a first detectable label and a sequence that binds the first nucleic acid molecule;
iii) detecting a signal from the first detectable label;
iv) reducing the signal of the first detectable label;
v) contacting the second nucleic acid molecule with a nucleic acid molecule comprising a second detectable label and a sequence that binds the second nucleic acid molecule; and
vi) detecting a signal from the second detectable label;
thereby sequentially detecting different target analytes in the sample.

In some embodiments, the first and second nucleic acid molecules are single stranded. In some embodiments, the sequence that binds the first nucleic acid molecule is complementary to a region of the unique sequence of the first nucleic acid molecule, and the sequence that binds the second nucleic acid molecule is complementary to a region of the unique sequence of the second nucleic acid molecule. In some embodiments, the nucleic acid molecule comprising the first detectable label forms a duplex along at least a portion of the first nucleic acid molecule, and the nucleic acid molecule comprising the second detectable label forms a duplex along at least a portion the second nucleic acid molecule. In some embodiments, the first and second detectable labels are the same or different.

In some embodiments, the method comprises repeating steps (iv)-(vi) for each additional target analyte immobilized on the solid support.

In some embodiments, reducing the signal of the detectable label comprises quenching, inactivating, or removing the signal or detectable label. In some embodiments, removing the signal comprises digesting the nucleic acid comprising the detectable label. In some embodiments, the digesting involves using a restriction enzyme and/or a DNA glycosylase combined with endonuclease VIII. In some embodiments, removing the signal involves photocleavage of the nucleic acid backbone comprising a photocleavable spacer. In some embodiments, removing the signal comprises displacing the nucleic acid strand comprising the detectable label. In some embodiments, displacing the nucleic acid strand includes using a polymerase enzyme that has a strand displacement function. In some embodiments, the displacing the nucleic acid strand comprises using toehold exchange strand displacement.

In some embodiments, the nucleic acid molecule comprising the detectable label forms a duplex along at least a portion of the first nucleic acid molecule, the second nucleic acid molecule, or both.

In some embodiments, the first nucleic acid molecule, the second nucleic acid molecule, or both, are attached to the binding agent via a 5′ phosphate group, an amine group, a carboxyl group, a hydroxyl group, a sulfhydryl group, click chemistry, copper(I)-catalyzed azide-alkyne cycloaddition (CuAAC), strain-promoted azide-alkyne cycloaddition (SPAAC), strain-promoted alkyne-nitrone cycloaddition (SPANC), or a linker. In some embodiments, the single-stranded nucleic acid molecule is attached via reductive amination following oxidation of carbohydrates on the binding agent. In some embodiments, a linker comprising biotin, streptavidin, protein A, protein G, protein A/G, or protein L is used to attach the single-stranded nucleic acid. In some embodiments, the nucleic acid molecule(s) comprising unique sequence(s) can be attached to the binding agent either covalently or non-covalently through an interaction between two or more molecules that specifically and stably associate.

In some embodiments, the binding agent comprises an antibody or antigen-binding fragment thereof, a nanobody, affibody or other antibody mimetic, an aptamer, a receptor, a ligand, a peptide, a lectin, a nucleic acid molecule, or a small molecule.

In another aspect, a composition comprising one or more binding agents attached to one or more target analytes is described herein. In some embodiments of the composition, the target analytes are immobilized on a solid support. In some embodiments, the binding agent is conjugated to a nucleic acid molecule comprising a unique sequence. In some embodiments, the nucleic acid molecule comprises a duplex along at least a portion of the nucleic acid molecule.

In some embodiments, the nucleic acid molecule comprises a first oligonucleotide attached to the binding agent and a second oligonucleotide comprising a detectable label hybridized to the first oligonucleotide.

In some embodiments, the first oligonucleotide is attached to the binding agent via a 5′ phosphate group, an amine group, a carboxyl group, a hydroxyl group, a sulfhydryl group, click chemistry, copper(I)-catalyzed azide-alkyne cycloaddition (CuAAC), strain-promoted azide-alkyne cycloaddition (SPAAC), strain-promoted alkyne-nitrone cycloaddition (SPANC), or a linker. In some embodiments, the single-stranded nucleic acid molecule is attached via reductive amination following oxidation of carbohydrates on the binding agent. In some embodiments, a linker comprising biotin, streptavidin, protein A, protein G, protein A/G, or protein L is used to attach the single-stranded nucleic acid. In some embodiments, the nucleic acid molecule(s) comprising unique sequence(s) can be attached to the binding agent either covalently or non-covalently through an interaction between two or more molecules that specifically and stably associate.

In some embodiments, the binding agent comprises an antibody or antigen-binding fragment thereof, a nanobody, affibody or other antibody mimetic, an aptamer, a receptor, a ligand, a peptide, a lectin, a nucleic acid molecule, or a small molecule.

In another aspect, a method for producing a composition described herein is provided. In some embodiments, the method comprises contacting a binding agent described herein to the target analyte. In some embodiments, the target analyte is immobilized on a solid support.

In another aspect, a kit comprising one or more compositions described herein is provided.

### BRIEF DESCRIPTION OF THE DRAWINGS

FIG. 1 shows a schematic of one embodiment of the method described herein. The unique nucleic acid molecules are attached to different binding agents through a biotin-streptavidin linkage.

FIG. 2 shows representative capture, probe, and quench oligonucleotides (SEQ ID NOs 1, 2 and 3, respectively) as described herein.

FIGS. 3A-3C show representative data of sequential multiplex Western blotting. FIG. 3A shows a representative sequential multiplex Western blot experiment using oligo encoded anti-PCNA mAb and anti-PARP mAb and a single membrane strip, as described in the Examples. FIG. 3B shows overlays of electropherogram data from adjacent images of the same sequential multiplex Western blot experiment, as described in the Examples. FIG. 3C shows traditional chemiluminescent Western blots as controls of the same PARP and PCNA targets, as described in the Examples.

FIG. 4 shows representative data of sequential multiplex western blotting using streptavidin-conjugated antibodies against human PCNA and GAPDH and detection with 5′-Cy5.5 labeled detection probe oligos.

FIGS. 5A and 5B show that the signal-to-noise ratio for detection of PCNA from a HEK293 lysate is stable over at least 10 probe-wash-detection-quench-wash cycles.

FIG. 6 shows that the detectable signal associated with the GAPDH binding agent can be reduced using a restriction enzyme digestion of the DNA duplex formed between the unique capture oligonucleotide and the probe oligonucleotide bearing the detectable label.

FIG. 7 shows that the solid support can be a magnetic particle and that the detectable signal associated with the human IL-6 binding agent can be reduced using a restriction enzyme or USER enzyme.

FIG. 8 demonstrates that the solid support can be a magnetic particle and that the detectable signal can be reduced using a toehold exchange strand displacement process.

### DEFINITIONS

Technical and scientific terms used in this disclosure have the meanings that are commonly recognized by those skilled in the art. See, e.g., Lackie, DICTIONARY OF CELL AND MOLECULAR BIOLOGY, Elsevier (4.sup.th ed. 2007); Green, M. R. and Sambrook J., MOLECULAR CLONING, A LABORATORY MANUAL, Fourth Edition, Cold Spring Harbor Lab Press (Cold Spring Harbor, N.Y. 2012). However, the following terms may have additional or alternative definitions, as described below, which are provided to facilitate understanding of certain terms used frequently herein and are not intended to limit the scope of the present disclosure.

The term “comprise” or “include” and variations thereof such as “comprises,” “comprising,” “includes,” and “including,” when referring to a step or an element, are intended to mean that the addition of further steps or elements is optional and not excluded. Any methods, devices, and materials similar or equivalent to those described herein can be used in the practice of the methods described herein.

As used herein, the term “binding agent” or “binding partner” refers to a molecule, complex, or assembly, that binds to another entity, such as a target analyte corresponding to and/or representing the presence or absence or abundance of the target. The binding agent can bind specifically to the entity, and thus may form a specific binding pair with the entity. Non-limiting examples of specific binding pairs include complementary nucleic acids, a receptor and its ligand, biotin and avidin/streptavidin, an antibody or fragment thereof and a corresponding antigen, an antibody and protein G, polyhistidine and Ni′, a transcription factor and a nucleic acid containing a binding site for the transcription factor, a lectin and its carbohydrate-bearing partner, or an aptamer and its partner. Non-limiting examples of molecules that can specifically interact with or specifically bind to a target molecule include nucleic acids (e.g., oligonucleotides), proteins (e.g., antibodies, transcription factors, zinc finger proteins, non-antibody protein scaffolds, receptors, ligands), peptides, aptamers and small molecules.

“Specific binding” with respect to a binding agent and a particular target (and/or with respect to a product corresponding to the particular target) in an assay refers to binding between the binding agent and the target (and/or the binding agent and the product) that is substantially exclusive of other targets (and/or their corresponding products) in the assay.

The term “solid support” refers to a surface that is capable of binding to an analyte, such as a membrane, the surface of a container (e.g., a well in a plate), a slide or coverslip, a channel or chamber such as in a microfluidic chip, a capillary, dipstick, lateral flow material, filter materials, or a particle such as a bead, microparticle or nanoparticle. The solid support can be treated with reagents that enhance binding of the analyte. The surface can also contain binding agents that specifically bind or capture the target analyte, for example an antibody or fragment thereof.

The term “sample” refers to a compound, composition, and/or mixture of interest, from any suitable source(s). A sample generally includes at least one target analyte that may be present in the sample. Samples may be analyzed in their natural state, as collected, and/or in an altered state, for example, following storage, preservation, extraction, lysis, dilution, concentration, purification, filtration, mixing with one or more reagents, partitioning, or any combination thereof, among others.

The sample may be of any suitable type for any suitable purpose. Clinical samples may include nasopharyngeal wash, blood, plasma, cell-free plasma, buffy coat, saliva, urine, stool, sputum, mucous, wound swab, tissue biopsy, milk, a fluid aspirate, a swab (e.g., a nasopharyngeal swab), and/or tissue, among others. Environmental samples may include water, soil, aerosol, and/or air, among others. Research samples may include cultured cells, primary cells, bacteria, spores, viruses, small organisms, any of the clinical samples listed above, or the like. Additional samples can include foodstuffs, weapons components, biodefense samples to be tested for bio-threat agents, and suspected contaminants.

Samples may be collected for diagnostic purposes (e.g., the quantitative measurement of a clinical analyte such as an infectious agent) or for monitoring purposes (e.g., to determine that an environmental analyte of interest such as a bio-threat agent has exceeded a predetermined threshold).

Biological samples can be obtained from or may contain any suitable biological organism(s), e.g., at least one animal, plant, fungus, bacterium, or other organism, or at least one portion thereof (e.g., one or more cells or proteins therefrom). In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue and/or bodily fluid obtained from an organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, transformed cells, and stem cells; stool, urine, etc. A biological sample can be obtained from a biopsy. A biological sample can also be obtained from a preserved or archived sample, e.g., an FFPE sample, samples stored in liquid nitrogen, or sample spotted and dried onto cards.

In some embodiments, the sample is an environmental sample, for example, an air, water, or soil sample. The sample can derive from a particular environmental source such as a particular lake, region, aquifer, watershed, or particular ecosystem or geographical area. Alternatively, the sample can be obtained from a swipe, scrape, etc. of an area, object, or space. For example, the same may be an air or water sample, or a swipe or scrape, from a hospital room, bed, or other physical object.

The sample can be prepared to improve efficient identification of a target. For example, the sample can be purified, fragmented, fractionated, homogenized, or sonicated. In some embodiments, one or more targets can be extracted or isolated from a sample (e.g., a biological sample). In some embodiments, the sample is enriched for the presence of the one or more targets. In some embodiments, the targets are enriched in the sample by an affinity method, e.g., immunoaffinity enrichment. For example, the sample can be enriched for biological particles/targets in general, or for particular types of particles/targets, by immunoaffinity, centrifugation, or other methods known in the art to capture and/or isolate particles/targets.

In some embodiments, the sample is enriched for targets using size selection (e.g., to remove small/short molecules and/or large/long molecules).

A “target” refers to an analyte of interest (or a region thereof). A target interchangeably may be termed an analyte. The target is typically detected by an assay, such as a multiplexed assay described herein, and may be contained by a sample. The target may be a molecule (a target molecule), or an assembly or complex of two or more molecules (a target assembly/complex). The target may be a portion (or all) of a molecule, or a portion (or all) of an assembly/complex. Exemplary targets include nucleic acids, nucleic acid sequences, proteins (e.g., an antibody, enzyme, growth factor, clotting factor, phosphoprotein, etc.), protein sequences (e.g., epitopes/haptens), carbohydrates, metabolites, and biological particles.

As used herein, “nucleic acid” refers to a molecule/assembly comprising a chain of nucleotide monomers. Nucleic acids with a natural structure, namely, deoxyribonucleic acid (DNA) and ribonucleic acid (RNA), generally have a backbone of alternating pentose sugar groups and phosphate groups. Each pentose group is linked to a nucleobase (e.g., a purine (such as adenine (A) or guanine (T)) or a pyrimidine (such as cytosine (C), thymine (T), or uracil (U))). Nucleic acids with an artificial structure are analogs of natural nucleic acids and may, for example, be created by changes to the pentose and/or phosphate groups of the natural backbone. Exemplary artificial nucleic acids include glycol nucleic acids (GNA), peptide nucleic acids (PNA), locked nucleic acids (LNA), threose nucleic acids (TNA), phosphorothioates, phosphoramidates, methyl phosphonates, chiral-methyl phosphonates, 2-O-methyl ribonucleotides, and the like.

The term “nucleic acid” includes DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, spacers with photocleavable bonds (for example, those from Integrated DNA Technologies (IDT)), unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore (e.g., quantum dot), fluorescence quenching agent, FRET acceptor or donor, biotin, or another moiety.

A single chain of a nucleic acid may be composed of any suitable number of nucleotides, such as at least 2, 5, 10, 20, 50, 100, 200, 500, or 1000 nucleotides, among others. Generally, the length of a nucleic acid chain corresponds to its source, with synthetic nucleic acids (e.g., primers and probes) typically being shorter, and biologically/enzymatically generated nucleic acids (e.g., nucleic acid analytes) typically being longer. “Nucleic acid” refers to a plurality of nucleic acids of different sequence, length, type, or a combination thereof, among others.

The sequence of a nucleic acid is defined by the order in which nucleobases are arranged along the backbone. This sequence generally determines the ability of the nucleic acid to bind specifically to a partner chain (or to form an intramolecular duplex) by hydrogen bonding. In particular, adenine pairs with thymine (or uracil), and guanine pairs with cytosine. A nucleic acid chain or region that can bind to another nucleic acid chain or region in an antiparallel fashion by forming a consecutive string of such base pairs with the other chain or region is termed “complementary.”

An “oligonucleotide” refers to a nucleic acid that is shorter than 500, 200, or 100 nucleotides in length. The oligonucleotide may be synthesized chemically, optionally without catalysis by an enzyme. Oligonucleotides may function, for example, as primers or probes.

A “detection reagent” refers to a reagent that facilitates or enables detection of the presence or absence and/or amount of a target analyte with a suitable detector (e.g., an optical detector). A set of detection reagents may be used in the methods described herein. The set of detection reagents may include at least one binding agent that binds specifically to only one of the targets to be assayed and/or that binds nonspecifically to each of the targets to be assayed. The binding partner may include a label and/or may be luminescent (and/or may have a luminescent form).

A “label” or “detectable label” refers to an identifying and/or distinguishing marker or identifier that is connected, attached or conjugated to, or integral with, a compound, target analyte, or nucleic acid described herein.

A molecule or other entity that is “attached” to a label (e.g., as for a labeled probe as described herein) is one that is attached covalently (“conjugated”) to the label by one or more chemical bonds, or attached noncovalently to the label, such as through one or more ionic, van der Waals, electrostatic, and/or hydrogen bonds such that the presence of molecule can be detected by detecting the presence of the label.

The terms “polypeptide,” “peptide” and “protein” are used interchangeably herein to refer to a polymer of amino acid residues. The terms apply to naturally occurring amino acid polymers, non-naturally occurring amino acid polymers, and amino acid polymers in which one or more amino acid residue is an artificial chemical mimetic of a corresponding naturally occurring amino acid.

The term “linker” refers to a compound that links or attaches two different molecules to each other. For example, the linker can include biotin and/or streptavidin, protein A, protein G, or protein A/G. Linkers can also include proteins or protein domains, both natural and synthetic, that covalently or non-covalently associate and/or combine (e.g., spy-catcher/spytag (see Hatlem D, et al., Catching a SPY: Using the SpyCatcher-SpyTag and Related Systems for Labeling and Localizing Bacterial Proteins. Int J Mol Sci. 2019; 20(9):2129. Published 2019 Apr. 30. doi:10.3390/ijms20092129); Profinity eXxact™ (Bio-Rad Laboratories)). Chemical linkers include carbohydrate linkers, lipid linkers, fatty acid linkers, nucleic acid linkers, and polyether linkers, e.g., PEG. For example, poly(ethylene glycol) linkers are available from Shearwater Polymers, Inc. Huntsville, Ala. The linkers can optionally have amide linkages, sulfhydryl linkages, or heterobifunctional linkages.

The term “unique sequence” refers to nucleic acid sequence that is different from (i.e., not the same as) other nucleic acid sequences. The unique sequence can be comprised in a single-stranded nucleic acid molecule that is attached to a binding agent described herein. The unique sequence can differ from other nucleic acid sequences by 1, 2, 3, 4, 5, 6, 7, 8, 9, 10 or more nucleotides or nucleobases.

### DETAILED DESCRIPTION OF THE INVENTION

### I. Introduction

The present application provides methods and compositions that are useful for sequentially detecting the presence of target analytes in a sample. The analytes in the sample can be immobilized on a solid support. The methods and compositions provide a solution to the problems associated with existing assays, and provide advantages over prior methods, such as fast, sensitive, gentle probing and detection of analytes with a high level of multiplexing that exceeds current capabilities with multicolor fluorescence detection. In the case of Western blotting, the method does not require stripping and reprobing, greatly accelerating multiplex detection of proteins on the blot from a multiple day process to a few hours. The method is compatible with chemiluminescence, fluorescence and other modes of detection. Another advantage is that the method can simplify and reduce instrument cost, as multiplex detection can be performed using a single color as the detectable label instead of using multiple lasers and filter sets as in current assays. The reduction in cost may lead to wider adoption of the method by scientists in the field. In addition, throughput can be further extended by combining the method with the use of traditional multicolor fluorescent dyes. Multicolor detections can also be used, for example, to include internal controls that are detected at each cycle on a separate imaging channel. Additional advantages include the use of single stranded nucleic acid molecules, such as oligonucleotides, which allow high level multiplexing from a single sample, reducing the need to normalize across lanes in a gel.

Described herein are methods and compositions for sequential multiplex western blotting and sequential multiplex ELISA assays. In some embodiments, the methods use binding agents attached to nucleic acid molecules comprising a detectable label. In some embodiments, the nucleic acid molecule attached to each binding agent comprises a unique sequence. In some embodiments, the membrane or Elisa well or plate is simultaneously contacted with one or more binding agents that are specific for the target analytes of interest, and then sequentially detected using a nucleic acid molecule comprising a detectable label. After imaging, the first detectable label is gently removed, quenched, or inactivated to reduce or eliminate its corresponding signal, and in some embodiments, the next analyte is concurrently probed. The cycle is repeated for each target analyte being evaluated.

In some embodiments, the target analytes are contacted with a binding agent that is attached or conjugated to nucleic acid molecules comprising a detectable label, detectable probe, or detectable moiety. In some embodiments, the labeled nucleic acid molecules are single stranded DNA or RNA molecules that are sequentially contacted with the target analytes in the sample. In some embodiments, the target analytes are first contacted with a binding agent comprising single stranded nucleic acid molecules that are complementary to the labeled single stranded nucleic acid molecules. After the first target analyte is detected, identified and/or quantitated, the detectable label can be quickly, selectively, and gently quenched, inactivated or removed allowing for detection of the next target analyte in a repetitive cycle without significant loss of antigen from the solid surface.

In some embodiments, the binding agent comprises a detection means, such as a detectable label described herein. In some embodiments, the method comprises a means for detecting target analytes in a sample, such as a means for detecting a binding agent comprising a detectable label bound to a target analyte described herein.

### II. Methods

Described herein are methods for sequentially detecting the presence of target analytes in a sample (e.g., a biological sample). In some embodiments, the method comprises contacting a sample comprising one, two or more (e.g., a plurality) target analytes with binding agents described herein. The target analytes can be immobilized on a solid support. The sample is contacted with one, two or more (e.g., a plurality) binding agents that specifically bind an analyte in the sample (referred to as analyte-specific binding agents). In some embodiments, the sample or solid support is contacted with a plurality of analyte-specific binding agents that specifically bind different analytes in the sample. In some embodiments, the sample or solid support is simultaneously contacted with a plurality of analyte-specific binding agents that specifically bind different analytes in the sample.

In some embodiments, the solid support is a surface, such as a membrane, surface of a multi-well plate, or a micro or nanoparticle. The solid surface can be blocked to prevent non-specific binding of the binding agents. In some embodiments, the solid surface is a membrane used in Western blot analysis, and the membrane is blocked with double-stranded DNA, tRNA, heparin sulfate, dextran sulfate, or anionic polymer.

In some embodiments, each of the analyte-specific binding agents is attached or conjugated to a nucleic acid molecule (e.g., a first nucleic acid molecule) comprising a unique sequence, such that each analyte-specific binding agent comprises a different unique sequence. For example, a binding agent that binds analyte A can be conjugated to a nucleic acid molecule comprising unique sequence A′, and a binding agent that binds analyte B can be conjugated to a nucleic acid molecule comprising unique sequence B′. In some embodiments, the nucleic acid molecule is covalently attached to the binding agent. In some embodiments, the nucleic acid molecule is non-covalently attached to the binding agent.

In some embodiments, the nucleic acid molecule attached or conjugated to a binding agent is a single stranded molecule, such as an oligonucleotide (also referred to as a “capture oligonucleotide”). The nucleic acid molecule can be DNA, RNA, or can comprise artificial nucleotides or analogs thereof. For example, the nucleic acid molecule can comprise locked nucleotides that are resistant to exo-nuclease activity.

In some embodiments, after each analyte-specific binding agent is attached to a nucleic acid molecule comprising the unique sequence, two or more different binding agents are pooled and simultaneously contacted with the sample. The sample can comprise analytes immobilized on a solid support.

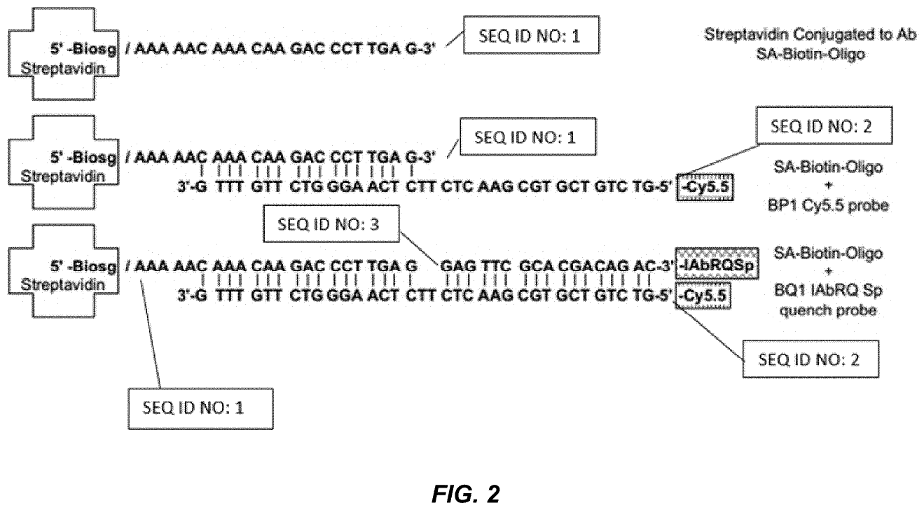

In some embodiments, the sample comprising the target analytes is then contacted with a nucleic acid molecule (e.g., a second nucleic acid molecule) comprising a nucleic acid sequence that is complementary to the unique sequence attached to an analyte-specific binding agent (e.g., a first analyte-specific binding agent) under conditions sufficient for hybridization between the complementary nucleic acid strands. In some embodiments, the second or complementary nucleic acid molecule comprises a detectable label or probe (also referred to as a “complementary probe oligonucleotide” or “probe oligonucleotide”). Hybridization of the complementary sequences results in the detectable label or probe being attached to the binding agent. In some embodiments, the nucleic acid molecule comprising the complementary nucleic acid sequence forms a duplex region with the unique sequence attached to an analyte-specific binding agent (for example, a duplex between the capture oligo and probe oligo). The duplex region can extend along only a portion of the nucleic acid molecule attached or conjugated to a binding agent, such that the nucleic acid molecule comprises a duplex region and single stranded region. In some embodiments, the single stranded region is located immediately 3′ of the detectable label or probe, as shown in FIG. 2.

The signal produced by the detectable label or probe attached to the binding agent is then detected using a method or system known in the art. For example, the label or probe can be imaged with a device that is capable of detecting the signal. In some embodiments, the label is a fluorescent label, and the signal can be detected with a device equipped with appropriate filters for measuring and quantitating fluorescent wavelengths emitted by the label or probe. Other examples include enzymes as labels, the products of which are detectable. Examples of enzyme labels include horseradish peroxidase (HRP), alkaline phosphatase, and beta-galactosidase. Enzyme labels can be conjugated to nucleic acid molecules attached to the binding agents described herein. Additional examples of detectable labels are described below.

After detecting the first target analyte in the sample (by detecting the detectable label attached to the first analyte-specific binding agent), the signal from the detectable label is reduced or eliminated. The signal can be reduced or eliminated using gentle methods that do not remove or reduce the amount of the target analytes on the solid support, i.e., methods that do not use harsh reagents such as detergents, reducing agents, or low pH, and/or do not use elevated temperatures (heat) between detection cycles. The method also does not require time consuming stripping and re-probing the solid support to remove the binding agent after detecting the detectable label. The method allows a single detectable label to be used to detect all the target analytes present in a sample (single color multiplexing), which greatly simplifies and reduces the cost of instruments required to detect multiple different labels for each target analyte.

In some embodiments, the signal from the detectable label is reduced by quenching, for example, using dynamic quenching and/or static quenching mechanisms. Examples of dynamic quenching include Förster resonance energy transfer or fluorescence resonance energy transfer (FRET), and Dexter electron transfer (also known as exchange or collisional energy transfer). Other examples of fluorescent quenchers include dark quenchers.

In some embodiments, the signal is quenched using a proximity-dependent pair of hybridization probes that exhibit FRET when bound adjacent to one another. In some embodiments, the signal is quenched using a quencher molecule attached to an oligonucleotide that hybridizes to the single stranded region located 3′ of the detectable probe. In some embodiments, the signal is quenched using a hairpin nucleic acid molecule comprising a fluorophore and a quencher, such as a Molecular Beacon probe (“beacon”). In some embodiments, the beacon is designed to anneal to the capture oligo, thereby unfolding and separating the fluorescent label and quencher molecule producing a detectable signal. Any beacon not bound to a target would have the fluorescent label on the opposite end quenched. To remove the signal, an unlabeled oligo that is also complementary to the capture oligo can be added, which displaces the bound beacon allowing the hairpin to reform and quenching its signal. The unlabeled oligo can be designed to bind more tightly/stably to the capture oligo to ensure the beacon is out-competed.

In some embodiments, the signal is reduced by contacting the nucleic acid comprising the detectable label with a restriction enzyme that cleaves or digests the nucleic acid to release the label, which is removed by washing. In some embodiments, the restriction enzyme is a four (4)-base cutter, such as CviQI or CviAII, which have maximal activity at ambient temperatures (available from New England Bio Labs), avoiding harsh conditions between detection cycles that may otherwise remove analyte from the surface).

In some embodiments, the capture and/or probe oligonucleotide contains one or more uracil bases, and the signal is reduced using USER™ (Uracil-Specific Excision Reagent) Enzyme, which generates a single nucleotide gap at the location of a uracil residue (available from New England Bio Labs). USER™ Enzyme is a mixture of Uracil DNA glycosylase (UDG) and the DNA glycosylase-lyase Endonuclease VIII. UDG catalyzes the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released. The USER™ enzyme effectively cleaves the oligonucleotide(s), either creating shorter strands that easily dissociate at ambient temperatures, or are directly released in the case of cleavage within single-stranded regions, thereby releasing the label, which can be washed away.

In some embodiments, the complementary nucleic acid molecule includes a photocleavable spacer. To reduce the signal from the detectable label, the photocleavable spacer can be exposed to long wavelength UV light, which hydrolyzes the nucleic acid backbone and releases the detectable probe.

In some embodiments, the signal from the detectable label is reduced by photobleaching (e.g., as described in Schubert W. et al. Nat. Biotech, 2006; 24:1270-78, which is incorporated by reference herein).

In some embodiments, the signal from the detectable label is reduced through a process of strand displacement. Examples of strand displacement are well known in the art and include toehold mediated strand displacement (Zhang, D Y et. al. 2012, Nature Chem 4, p 208-214, “Optimizing the specificity of nucleic acid hybridization.”; Pallikkuth, S, et. al. 2018, PLOS One, 1-11, “Sequential super-resolution imaging using DNA strand displacement.”) and RNA/DNA polymerase mediated strand displacement activity. In some cases, a separate complementary oligonucleotide (toehold oligo) is partially annealed to a single stranded region of the capture or probe oligonucleotide, and which then subsequently migrates along an adjacent a region forming a new duplex that displaces and releases the original probe oligonucleotide from capture oligonucleotide so that it can be washed away. If the single stranded toehold region was on the probe oligonucleotide the unoccupied capture oligo can be regenerated such that the same analyte can be probed multiple times if desired. In some embodiments, a primer oligo can be annealed to a single stranded region of the capture or probe oligonucleotide, and by using a polymerase and nucleotides, the probe oligo displaced and able to be washed away. In other embodiments, the probe oligo can contain a hairpin with a 3′ end, such that a separate primer is not required for the polymerase to extend the sequence and displacing the label.

After the signal from the label attached to the first binding agent is reduced or eliminated, another analyte in the sample can be detected. In some embodiments, the sample is contacted with another (different or third) nucleic acid molecule comprising a nucleic acid sequence that is complementary to the unique sequence attached to a different analyte-specific binding agent (e.g., a second analyte-specific binding agent) under conditions sufficient for hybridization between the complementary nucleic acid strands. As above, in some embodiments, the complementary nucleic acid molecule comprises a detectable label or probe. The detectable label or probe can be the same or different than the detectable label or probe attached to the other binding agents (or the other complementary nucleic acid molecules) in the assay.

In some embodiments, the first detectable label is quenched and the sample is contacted with the next complementary nucleic acid molecule simultaneously or concurrently.

The above steps can be repeated to detect additional analytes in the sample, thereby resulting in sequential detection of target analytes in the sample.

In some embodiments, the detectable label comprises an enzyme based detection reagent. In these embodiments, the enzymes can be inactivated by inhibitors, such as irreversible inhibitors.

In some embodiments, capture oligos can be made to resist nuclease degradation, while detection oligos can be made susceptible to nuclease hydrolysis, thereby releasing the probe label upon degradation.

In some embodiments, the capture oligo is attached to the binding agent with biotin through the 5′ end. The capture oligo can also be attached to the binding agent via its 3′ end (for example, using biotin-SA or directly attached to the binding agent). To reduce the signal from the detectable label, the probe oligo comprising a detectable label is annealed forming a single stranded region at its 5′ end. The detectable label can be removed by annealing a primer oligo to the single stranded region of the probe oligo (analogous to annealing the quench oligo in FIG. 2), and then adding a DNA Polymerase and dNTPS to extend the primer, thereby releasing the detection oligo, which cannot bind to the duplex strand comprising the capture oligo. In some embodiments, the capture oligo can be degraded by 5′->3′ exonuclease activity or strand-displacement depending on the polymerase used.

In some embodiments, the CRISPR system can be adapted to cleave or displace the probe strand.

### III. Binding Agents

The binding agents that bind specific target analytes in the sample can include proteins (e.g., antibodies, transcription factors, zinc finger proteins, non-antibody protein scaffolds, receptors, ligands, receptor-ligand pairs), lectins directed against different carbohydrates, peptides, peptide aptamers, nucleic acid aptamers, and small molecules. Additional examples of binding agents can be found in a protein binding database (e.g., The Binding Database, bindingdb.org) that lists thousands of protein targets and small molecules.

In some embodiments, the binding agent is an antibody or antigen binding fragment thereof that specifically binds a target analyte (e.g. an antigen) in the sample. Examples of antibodies and antigen binding fragments include immunoglobulin molecules of any isotype (e.g., IgG and IgM molecules), Fab, diabodies (e.g., a heavy chain variable domain on the same polypeptide as a light chain variable domain, which are connected via a short peptide linker), Fab′, F(ab′)2, Fv domain antibodies and single-chain antibodies (e.g., scFv molecules). In some embodiments, the antibody is a “chimeric” antibody comprising portions from two different antibodies. Antibodies can comprise two full-length heavy chains and two full-length light chains, or derivatives, variants, or fragments thereof, or can comprise only heavy chains, such as antibodies produced in camelids. Other examples include polyclonal antibodies, monoclonal antibodies, bispecific antibodies, minibodies, domain antibodies, synthetic antibodies (“antibody mimetics”), humanized antibodies, human antibodies, peptibodies and antigen binding fragments thereof.

### IV. Nucleic Acids

The binding agents described herein can be attached or conjugated to a nucleic acid molecule. The nucleic acid molecule can comprise DNA, RNA, single-stranded, double-stranded, or more highly aggregated hybridization motifs, and any chemical modifications thereof. In some embodiments, the nucleic acid molecule is a single stranded molecule.

In some embodiments, the nucleic acid includes chemical modifications. Examples of chemical modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore (e.g., quantum dot), quencher, biotin, or another moiety.

In some embodiments, the nucleic acid molecule can comprise an artificial structure or analogs of natural nucleic acids (e.g., non-natural nucleic acids). Exemplary artificial nucleic acids include glycol nucleic acids (GNA), peptide nucleic acids (PNA), locked nucleic acids (LNA), threose nucleic acids (TNA), phosphorothioates, phosphoramidates, methyl phosphonates, chiral-methyl phosphonates, and 2-O-methyl ribonucleotides. In some embodiments, the nucleic acid is blocked at the 5′ or 3′ end to prevent or inhibit exonuclease degradation.

In some embodiments, the nucleic acid molecule is attached or conjugated to a detectable label described herein.

In some embodiments, the nucleic acid molecule is less than about 100 nucleotides in length, for example 10-90, 10-80, 10-70, 10-50, 10-40, 15-90, 15-80, 15-70, 15-60, 15-50 or 15-40 nucleotides in length. The nucleic acid is generally designed to allow for stable annealing at the temperature used for hybridization between single strands. In some embodiments, the temperature is ambient temperature (e.g., 20-25° C.). Software known and available in the art can be used to design the nucleic acid sequences and predict dimers, hairpins and stability in different buffers, temperatures and salt conditions.

### V. Conjugation of Nucleic Acids to Binding Agents

The nucleic acids described herein can be conjugated to the binding agents using methods described in G. T. Hermanson, Bioconjugate Techniques, Third Edition, Academic Press (2013); Maerle A. V. et al., “Development of the covalent antibody-DNA conjugates technology for detection of IgE and IgM antibodies by immuno-PCR,” PLoS One. 2019; 14(1): e0209860; and Shahi, P. et al., Scientific Reports, 7:44447 “Abseq: Ultrahigh-throughput single cell protein profiling with droplet microfluidic barcoding;” which are incorporated by reference herein. Commercial oligonucleotide conjugation kits like THUNDER-LINK® PLUS OLIGO CONJUGATION SYSTEM from Expedeon can also be used.

In some embodiments, the single-stranded nucleic acid molecule (e.g., capture oligonucleotide) is attached to the binding agent via a 5′ phosphate group, an amine group, a carboxyl group, a hydroxyl group, or a sulfhydryl group. Sulfhydryl-reactive chemical groups include haloacetyls, maleimides, aziridines, acryloyls, arylating agents, vinylsulfones, pyridyl disulfides, TNB-thiols and disulfide reducing agents. Many sulfhydryl-reactive chemical groups conjugate to sulfhydryls by alkylation (e.g., the formation of a thioether bond) or disulfide exchange (formation of a disulfide bond).

In some embodiments, the nucleic acid molecule is conjugated to the binding agent using carbodiimide crosslinker chemistry, where carboxyl-reactive chemical groups are crosslinked to carboxylic acids (—COOH), which occur in proteins and many other biomolecules. Carbodiimide compounds such as EDC and DCC can be used to crosslink carboxylic acids to primary amines via amide bond formation. Sulfo-NHS (N-hydroxysulfosuccinimide) modification can also be used for converting carboxyl groups to amine-reactive NHS esters for conjugation of nucleic acids to binding agents described herein.

In some embodiments, the single-stranded nucleic acid molecule (e.g., capture oligonucleotide) is attached to the binding agent using click chemistry methods, such as copper(I)-catalyzed azide-alkyne cycloaddition (CuAAC), strain-promoted azide-alkyne cycloaddition (SPAAC), strain-promoted alkyne-nitrone cycloaddition (SPANC).

In some embodiments, the nucleic acid is conjugated to the binding agent using a suitable linker. Suitable linkers include, without limitation, biotin, streptavidin, protein A, protein G, protein A/G, and protein L. In some embodiments, the linker comprises biotin and/or avidin or streptavidin (SA). For example, the binding agent, such as an antibody, can be conjugated to SA, and the nucleic acid can be conjugated to biotin, or vice versa. In some embodiments, the linker is a chemical linker, such as a homo or heterobifunctional linker.

In some embodiments, the binding agent is conjugated to a linker using a commercially available kit, such as the LYNX Rapid & Rapid Plus Conjugation Kits® (Bio-Rad).

### VI. Detectable Labels

The binding agents or nucleic acids described herein can be attached to a detectable “label.” The label can be detectable by any suitable approach, including spectroscopic, photochemical, biochemical, immunochemical, chemical, or other physical methods. Suitable labels include fluorophores, chemiluminescence reactions, horse radish peroxidase (HRP), luminophores, chromophores, radioisotopes (e.g., ³²P, ³H), electron-dense reagents, enzymes, and specific binding partners. Methods of attaching detectable labels to binding agents are well known. For example, reviews of common protein labeling techniques are available in Biochemical Techniques: Theory and Practice, John F. Robyt and Bernard J. White, Waveland Press, Inc. (1987); R. Haugland, Excited States of Biopolymers, Steiner ed., Plenum Press (1983); Fluorogenic Probe Design and Synthesis: A Technical Guide, PE Applied Biosystems (1996); and G. T. Hermanson, Bioconjugate Techniques, Third Edition, Academic Press (2013), all of which are incorporated by reference herein.

The binding agents or nucleic acids described herein can be covalently attached (“conjugated”) to the label by one or more chemical bonds, or attached non-covalently to the label, such as through one or more ionic, van der Waals, electrostatic, and/or hydrogen bonds such that the presence of molecule can be detected by detecting the presence of the label.

The detectable label can have any suitable structure and characteristics. For example, a label can be a probe including an oligonucleotide and a luminophore associated with the oligonucleotide (e.g., with the luminophore conjugated to the oligonucleotide), to label the oligonucleotide. The detectable label can also be a pDot (polymer dot) which has an extremely bright and stable signal. The probe can also include an energy transfer partner for the luminophore, such as a quencher or another luminophore. Exemplary labeled probes include Eclipse™ probes, molecular beacon probes, proximity-dependent pairs of hybridization probes that exhibit FRET when bound adjacent to one another, or Dual Hybridization Probes.

In some embodiments, the signal from the detectable label is reduced or eliminated. The signal can be reduced or eliminated by quenching the signal (e.g., light emission from a fluorophore) in a proximity-dependent fashion. In some embodiments, light from the fluorophore is detected when the associated oligonucleotide (attached to fluorophore) binds to the complementary nucleic acid strand. The signal can be reduced by hybridizing a complementary oligonucleotide attached to a quencher molecule to the single stranded nucleic acid attached to the fluorescent probe, such that the quencher molecule and the fluorescent probe are in close proximity. In some embodiments, the detectable label is quenched (undetectable) until the nucleic acid molecule binds the complementary nucleic acid strand, and upon binding the signal can be detected. The quencher may be the same or different for each fluorophore. In some embodiments, the quencher molecule is IAbRQSp or a blackhole quencher. In other embodiments, the signal is reduced or eliminated by cleaving the nucleic acid attached to the detectable label, for example by digesting the nucleic acid with a restriction enzyme as described above. In other embodiments, the signal is reduced or eliminated through strand displacement such as a toehold-mediated or polymerase-mediated process.

In some embodiments, the detectable label comprises HRP. In some embodiments, HRP is conjugated to the complementary nucleic acid that hybridizes to the nucleic acid molecule attached to the binding agent.

In some embodiments, the label comprises or is attached to a photocleavable spacer.

In some embodiments, two or more labels (e.g., a first label, second label, etc.) combine to produce a detectable signal that is not generated in the absence of one or more of the labels. For example, in some embodiments, each of the labels is an enzyme, and the activities of the enzymes combine to generate a detectable signal. Examples of enzymes combining to generate a detectable signal include coupled assays, such as a coupled assay using hexokinase and glucose-6-phosphate dehydrogenase; and a chemiluminescent assay for NAD(P)H coupled to a glucose-6-phosphate dehydrogenase, beta-D-galactosidase, or alkaline phosphatase assay. See, e.g., Maeda et al., J Biolumin Chemilumin 1989, 4:140-148.

### VII. Compositions

Also provided are compositions comprising the binding agents described herein. In some embodiments, the composition comprises one or more binding agents attached to one or more target analytes immobilized on a solid support. The target analyte(s) can be immobilized on a solid support either directly or indirectly. For example, the target analyte(s) can be directly attached (immobilized) to the solid support, or indirectly attached to the solid support. In some embodiments, the target analyte(s) is indirectly attached to the solid support using an antibody immobilized on the solid support, and the analyte binds to the antibody, resulting in indirect immobilization of the analyte on the solid support. In some embodiments, the binding agent is conjugated to a nucleic acid molecule comprising a unique sequence and a detectable label. In some embodiments, the nucleic acid molecule comprises a duplex along at least a portion of the nucleic acid molecule. In some embodiments, the nucleic acid molecule comprises a first single stranded nucleic acid molecule (e.g. a first oligo or capture oligo) attached to the binding agent, and a second single stranded nucleic acid molecule comprising a detectable label (e.g., a second oligo or probe oligo) hybridized to the first oligonucleotide. In some embodiments, the one or more binding agents, or each of the binding agents, is/are attached to different first single stranded nucleic acid molecules, each comprising a unique sequence. For example, each binding agent can be attached to a different capture oligo comprising a unique sequence.

In some embodiments of the composition, the first single stranded nucleic acid molecule or oligonucleotide is attached to the binding agent via a 5′ phosphate group, an amine group, a carboxyl group, a hydroxyl group, or a sulfhydryl group. In some embodiments, the first single stranded nucleic acid molecule or oligonucleotide is attached to the binding agent using click chemistry, such as copper(I)-catalyzed azide-alkyne cycloaddition (CuAAC), strain-promoted azide-alkyne cycloaddition (SPAAC), or strain-promoted alkyne-nitrone cycloaddition (SPANC).

In some embodiments, the first single stranded nucleic acid molecule or oligonucleotide is attached to the binding agent using a suitable linker. Suitable linkers include, without limitation, biotin, streptavidin, protein A, protein G, protein A/G or protein L. In some embodiments, the binding agent is conjugated to streptavidin, and the first oligonucleotide comprises biotin that binds to the streptavidin, thereby linking the first oligonucleotide to the binding agent.

The composition can also include a first set of single stranded nucleic acid molecules (e.g., a set of capture oligos), wherein each member of the set comprises a different, unique sequence. In some embodiments, each analyte-specific binding agent is attached to a different member of the set of first single stranded nucleic acid molecules (e.g., a set of capture oligos), wherein each member of the set comprises a different, unique sequence.

The composition can also include a second set of single stranded nucleic acid molecules (e.g., a set of probe oligos), wherein each member of the set comprises a sequence that hybridizes (or is capable of hybridizing) to a member of the first set of single stranded nucleic acid molecules (e.g., the set of capture oligos) under appropriate conditions.

In some embodiments, the binding agent comprises an antibody or antigen binding fragment thereof, an aptamer, a receptor, a ligand, a peptide, or a small molecule.

Also described are methods for producing the compositions described herein. In some embodiments, the method comprises contacting one or more binding agents described herein to one or more cognate target analytes, wherein the one or more target analytes are immobilized, either directly or indirectly, on a solid support.

The analyte can be immobilized either directly or indirectly to the solid support. An example of indirect immobilization comprises a sandwich-type arrangement, where an antibody that is capable of binding the analyte is immobilized to a solid support. After contacting the sample comprising one or more target analytes to the solid support, the immobilized antibody binds to the analyte. The binding agent with the unique sequence can then bind to the same analyte, but at a different location on the analyte.

### VIII. Kits

Also provided are kits comprising the compositions described herein. For example, the kit can comprise one or more binding agents that bind (or are capable of binding) one or more target analytes in a sample (“analyte-specific binding agents”). In some embodiments, the kit includes one or more analyte-specific binding agents attached to a single stranded nucleic acid molecule, and/or reagents for attaching single stranded nucleic acid molecules to the analyte-specific binding agents, wherein the single stranded nucleic acid molecule (e.g., “capture oligo”) comprises a unique sequence. In some embodiments, the kit includes a set of first single stranded nucleic acid molecules (e.g., a set of capture oligos), wherein each member of the set comprises a different, unique sequence. In some embodiments, each analyte-specific binding agent is attached to a different single stranded nucleic acid molecule comprising a unique sequence. In some embodiments, each analyte-specific binding agent is attached to a different member of the set of first single stranded nucleic acid molecules (e.g., a set of capture oligos), wherein each member of the set comprises a different, unique sequence. The kit can further include complementary (e.g., second) single stranded nucleic acid molecule(s), or a set of complementary (e.g., second) single stranded nucleic acid molecules, that comprise a sequence complementary to the first single stranded nucleic acid molecule(s) attached to each binding agent, as described above. The complementary (e.g., second) single stranded nucleic acid molecule(s), or set of complementary (e.g., second) single stranded nucleic acid molecules, can comprise a detectable label described herein (e.g., “probe oligos”).

The kit can further include reagents for conjugating nucleic acids molecules to binding agents, and/or for conjugating detectable labels to nucleic acid molecules. In some embodiments, the kit can include a third oligo set for use as quenchers, or as primers for removing the detectable label.

### EXAMPLES

### Example 1

This Example describes the sequential detection of target proteins in a Western assay.

Methods:

Streptavidin Conjugation to Antibodies. Anti-hGAPDH, hPCNA, and hPARP Antibodies (obtained from Bio-Rad; see Table 1 below) were concentrated and washed twice with 1× Phosphate Buffered Saline (PBS), pH 7.4. Streptavidin was conjugated to each antibody using the Lynx Rapid Streptavidin Conjugation Kit® (Bio-Rad, # LNK161STR) and 100 μg of Ab per manufacturer instructions. 10 μl of Modifier reagent was added to each 100 μl of Ab solution and the volume was transferred to a lyophilized vial of streptavidin. A control sample (10 μl) was removed and immediately added 1 μl of Quench reagent. The reaction was incubated at room temperature (RT) for 3 hr, and then 10 μl of Quench reagent was added to each reaction and incubated for 30 min. The reactions were analyzed using Experion Pro260 assay under reducing and non-reducing conditions, to assess the level of conjugation. Final antibody-SA concentrations were 0.5-1.0 mg/ml.

TABLE 1

| Antibody | | Target MW | | | | Conc | Vol Used | Label Color |
|---|---|---|---|---|---|---|---|---|
| Target | Species | (kDa) | Vendor | Catalog# | Lot# | (ug/ul) | (ul) | Code |
| human | mouse | 37 | Bio-Rad | VMA00046 | 160822 | 0.5 | 200 | yellow |
| GAPDH | | | | | | | | |
| human | mouse | 29 | Bio-Rad | VMA00016XZX, | 1807 | 1 | 100 | blue |
| PCNA | | | | no | | | | |
| | | | | preservative | | | | |
| human | mouse | 116 | Bio-Rad | VMA00018XZX, | 1807 | 1 | 100 | green |
| PARP | | | | no | | | | |
| | | | | preservative | | | | |

| Materials | Vendor | Catalog# | Lot# |
|---|---|---|---|
| 0.5 ml PES concentrator, 30k MWCO | Thermo/Pierce | 88502 | TF268588A |
| 1X PBS, pH 7.4 Buffer | n/a | | |
| Lynx rapid streptavidin Ab conjugation Kit | Bio-Rad | LNK161STR | 180626 |

Western Blot Membrane Prep.

HEK293 lysate (reconstituted to 1 mg/ml in 1× Laemmli+40 mM DTT) was heated at 100° C. for 5 min. 10 μg (or 4 μl) of lysate was loaded onto a TGX 4-20% gel, and a Dual Color Precision Protein Standard was loaded in adjacent lanes. The gel was electrophoresed at 250 Volts for 22 min, then transferred to a PVDF membrane using TBT (7 min at 1.3 A). The membrane was blocked for 60 min in 1×PBST, 3% BSA, then blocked for 30 min in 1×PBST (0.1%), 1% BSA, 100 μg/ml sheared salmon sperm DNA (Thermo), 5 mM EDTA.

Sequential Multiplex Western Blotting Protocol

Separate Antibody-Streptavidin(SA)-biotin-capture oligo mixes were prepared and incubated 30 min at RT. Each mix contained:

a. 4 μl of Antibody-Streptavidin Conjugate, 4 ug Ab, 0.027 nmol Ab.

i. assume 1-2 SA/IgG=0.11-0.22 nmol biotin binding sites.

b. 2 μl of 100 μM Biotinylated-Capture oligo, 0.2 nmol.

c. 2 μl TE buffer, pH 7.5.

All the Ab-SA-biotin-CaptureOligo mixes were combined into 5 ml Ab Diluent Buffer (1×PBS, 1% BSA, 0.1% Tween 20, 5 mM EDTA, 50 μg/ml sheared salmon sperm dsDNA). Solution was added to the blot membrane in a square petri dish and incubated 0/N at 4° C. with rocking. The blots were washed 4 times with 10 ml Wash Buffer (1×PBS, 0.1% Tween 20, 5 mM EDTA) for 5 min each wash. The membrane was probed with 50 nM BP1 Detection probe oligo 1 (5′-Cy5.5 labeled) in 5 ml Probe Buffer, and incubated for 15 min at RT with rocking. The blots were washed 4 times with 10 ml Wash Buffer for 5 min each wash. The membrane was imaged using ChemiDoc Touch Cy5.5 channel. 50 nM Quench Probe 1 (BQ1) and 50 nM Detection Probe 2 (BP2) were combined into 5 ml Probe Buffer (1×PBS, 1% BSA, 0.1% Tween 20, 5 mM EDTA, 5 μg/ml sheared salmon sperm dsDNA, and incubated with the membrane for 15 min at RT to simultaneously quench the BP1 probe and detect the antigen target bearing the capture probe 2 using the BP2 probe. The blots were then washed as above. Strips of the blot were re-imaged on ChemiDoc Touch Cy5.5 channel. The steps were repeated to detect each additional target.

Results:

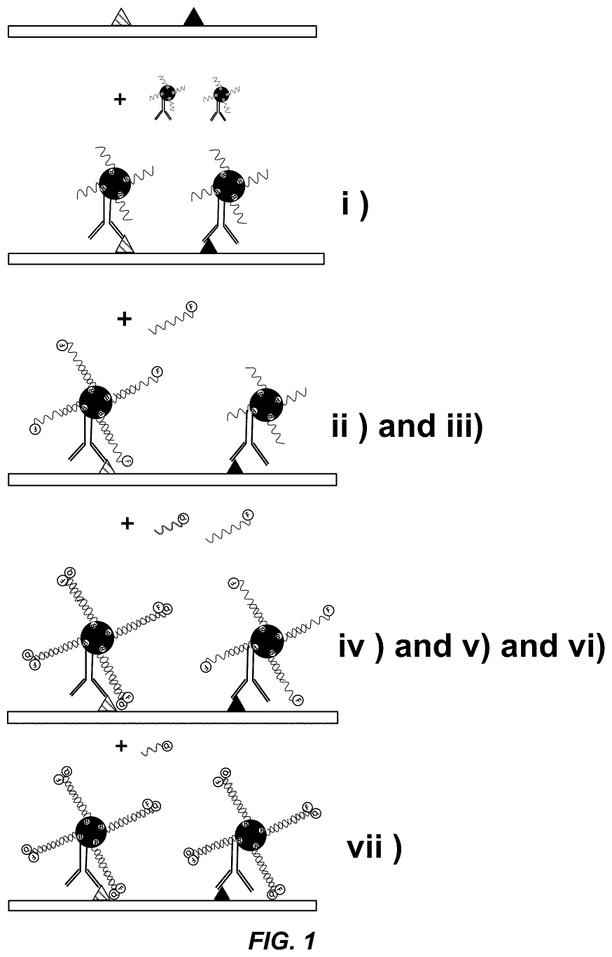

A representative method is illustrated in FIG. 1. Primary antibodies (Ab's) were conjugated to streptavidin (SA) using the LYNX Rapid Streptavidin Antibody Conjugation Kit (Bio-Rad) resulting in 1-3 SA:Ab ratio. A unique biotin-capture oligo was mixed with the SA-conjugated primary antibodies in separate tubes and incubated for 30 mins, using a 1:1 molar ratio (or slight excess) of biotin-oligo to SA sites. The membrane was blocked for 30-60 min as described under Methods.

The primary antibodies were then pooled, diluted in 5 mL Ab Diluent Buffer as a second blocker, and incubated 2 hrs at RT or overnight at 4° C.

The first Ab (bound to the first target analyte) was detected by incubating the blot with a complementary probe-oligo in 5 mL Probe Buffer for 15 min at room temperature. The blot was washed then imaged as described under Methods. The second Ab (bound to the second target analyte) was detected and the signal of the detectable label bound to the first target Ab was simultaneously quenched by incubating the blot for 15 min at room temperature with a complementary probe-oligo to the second capture oligo and a quench-oligo to the first probe-oligo. The blot was washed and imaged. The cycle was repeated for each subsequent antibody-target analyte binding pair tested.

Table 2 shows representative capture-probe and quench oligos.

| | | | | | | Label |
|---|---|---|---|---|---|---|
| | | SEQ ID | | | Label | Reduction |
| ID | Sequence (5′ → 3′) | NO: | Type | Label | Position | Method(s) |
| BC1 | biotin-AAAAACAAACAAGACCCTTGAG | 1 | capture | biotin | 5′ | |
| BP1 | Cy5.5- | 2 | probe | Cy5.5 | 5′ | |
| | GTCTGTCGTGCGAACTCTTCTCAAGGGTCTT | | | | | |
| | GTTTG | | | | | |
| BQ1 | GAGTTCGCACGACAGAC- IAbRQSp | 3 | quench | IAbRQSp | 3′ | |
| BC2 | biotin-CATACCCGTAATAGCGT | 4 | capture | biotin | 5′ | |
| BP2 | Cy5,5- | 5 | probe | Cy5.5 | 5′ | n/a |
| | ATGCGAATAATTGGTTTACGCTATTACGGGT | | | | | |
| | ATG | | | | | |
| BQ2 | ACCAATTATTCGCAT- IAbRQSp | 6 | quench | IAbRQSp | 3′ | |
| BC4B | biotin-CATACCTGTACCGTAATAGCGT | 7 | capture | biotin | 5′ | |
| BP4B | Cy5.5- | 8 | probe | Cy5.5 | 5′ | CviQI |
| | ATGCGAATAATTGGTTTACGCTATTACGGTA | | | | | (G\hat{ }TAC) |
| | CAGGTATG | | | | | |
| BQ2 | ACCAATTATTCGCAT- IAbRQSp | 6 | quench | IAbRQSp | 3′ | |
| BP4D | DTPA- | 9 | probe | dithiol-HRP | 5′ | CviQI |
| | ATGCGAATAATTGGUTUACGCUATTACGGT | | | | | (G\hat{ }TAC), |
| | ACAGGTATG | | | | | USER |
| BC15-2 | biotin-TTGCATCCAGTACTTCAATAC | 10 | capture | biotin | 5′ | |
| BP15A | Cy5.5- | 11 | probe | Cy5.5 | 5′ | CviQI |
| | AAAAAGACGTAGGGTATTGAAGTACTGGAT | | | | | (G\hat{ }TAC) |
| | GCAA | | | | | |
| RP15-2 | TTGCATCCAGTACTTCAATACCCTACGTC | 12 | toehold | n/a | n/a | toehold, |
| | | | | | | strand |
| | | | | | | exchange⁽¹⁾ |
| LRA | TTTCGTTTGCCTGCTTTATCTCTGTTCTACTAT | 13 | toehold | n/a | n/a | off target |
| | TTCCG | | | | | control⁽²⁾ |

Reference:
⁽¹⁾Zhang, DY et. al. 2012, Nature Chem 4, p208-214. Optomizing the specificity of nucleic acid hybridization.
⁽²⁾Pallikkuth, S, et. al. 2018, PLOS One, 1-11. Sequential super-resolution imaging using DNA strand displacement.

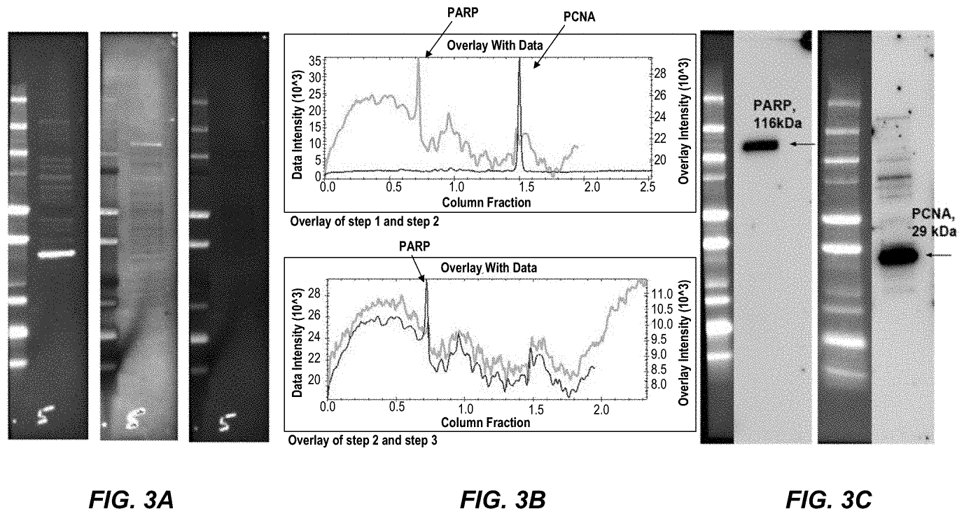

FIGS. 3A-3C show representative results for the Sequential Multiplex Western Blotting assay. FIG. 3A shows images from a representative sequential multiplex Western blot experiment using oligo encoded anti-PCNA mAb and anti-PARP mAb and a single membrane strip. Precision Plus Protein Dual Color Standards (Bio-Rad Laboratories, 4 ul) and HEK293 lysate (VLY001, Bio-Rad Laboratories, 10 ug) were loaded into alternating lanes of a 4-20% mini-PROTEAN TGX gel (Bio-Rad Laboratories), separated by SDS-PAGE, and transferred to a PVDF membrane using Trans-Blot Turbo (Bio-Rad Laboratories). The membrane was blocked with 1×PBS, 0.1% Tween20, 3% BSA for 60 min, followed by blocking for 30 min with 1×PBS, 0.1% Tween20, 1% BSA, 5 mM EDTA, 100 ug/mL sheared salmon sperm DNA (Thermo-Fisher Scientific). After blocking, the membrane was cut into 5 strips containing 1 standard and 1 lysate lane.

Mouse monoclonal antibodies used for fluorescence detection of PCNA (VMA00016, 29 kDa) and PARP(VMA00018, 116 kDa) were from Bio-Rad Laboratories and were first labeled with Streptavidin (SA) using the LYNX rapid streptavidin antibody conjugation kit also from Bio-Rad Laboratories. In separate tubes, 4 ug of each antibody was mixed with an approximately equimolar amount (based on number of biotin binding sites) of unique biotinylated-capture oligo. The antibodies-SA-oligo complexes were then diluted together in 5 ml of antibody diluent containing 1×PBST, 1% BSA, 5 mM EDTA, 50 ug/ml DNA, and incubated overnight at 4 C with gentle rocking.

The next day the membrane strip was washed with 1×PBST, 5 mM EDTA. Sequential probing of the strip was performed by first annealing 50 nM of cy5.5-labeled probe oligo (BP2) to the corresponding capture oligo (BC2) on the PCNA mAb by incubating for 15 min at RT in 5 mL wash buffer containing 5 ug/mL salmon DNA (Probe Diluent). The membrane strip was washed 4×5 min again in PBST, 5 mM EDTA and imaged using the Cy5.5 channel of ChemiDoc Touch Imager (Bio-Rad) (left side strip image) to detect PCNA. After imaging, 50 nM each of probe oligo BP1 and quench oligo BQ2 were added into 5 mL Probe Diluent and incubated for 15 min to simultaneously quench the signal of BP2 and detect the signal associated with annealing the BP1 cy5.5-labeled oligo to the complementary BC1 capture oligo attached to the PARP mAb via the streptavidin conjugate. After additional wash steps, the strip was reimaged, where the PCNA signal (BP2) was shown to be quenched and the PARP signal (BP1) was observed (middle strip image). Finally, addition of 50 nM BQ1 Quench oligo in 5 ml of Probe Diluent, washing and imaging resulted in the right side strip image where the PARP signal is quenched.

FIG. 3B shows line profile overlays (generated using ImageJ) of the left and middle strips (top) and the middle and right strips (bottom).

FIG. 3C shows images of controls to confirm that the primary antibodies could detect the targets of expected molecular weight when using a traditional western blot protocol and chemiluminescence (Clarity substrate, Bio-Rad Laboratories). Unconjugated mouse anti-PARP and mouse anti-PCNA primary antibodies (1:1000 dilution, bug in 10 ml 1×PBST, 3% BSA) plus Goat-anti-mouse-HRP (1:10,000 dilution in 1×PBST, 3% BSA) were used to detect the same targets in other strips from the same blot.

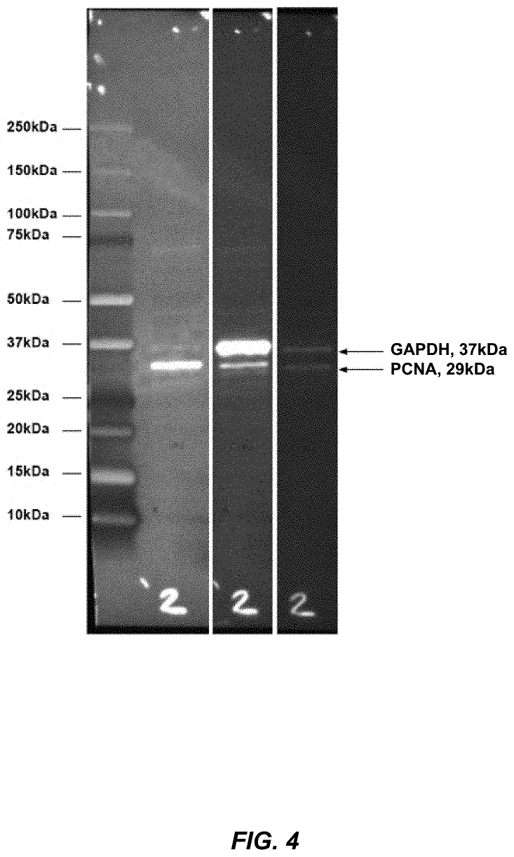

FIG. 4 shows the results from a representative sequential multiplex Western blot experiment using oligo encoded anti-PCNA mAb and anti-GAPDH mAb (VMA00046, Bio-Rad, 37 kDa) and a single membrane strip. In this experiment, the blot was incubated with 4 ug of anti-PCNA-SA-BC1oligo plus anti-GAPDH-SA-BC2oligo in 5 ml Probe Diluent overnight at 4 C. Note that in this experiment the PCNA-SA antibody contains the BC1 capture oligo rather than the BC2 capture oligo used in the previous experiment. PCNA was detected by incubating the blot for 15 min with 50 nM cy5.5-labeled BP1 oligo (left-side image). Subsequently, GAPDH was detected by incubating the same strip with 50 nM cy5.5-labeled BP2 oligo and 50 nM BQ1 Quench oligo for 15 min to simultaneously quench the PCNA signal and detect the GAPDH signal (middle image). A final 15 min incubation of the same blot with BQ2 quench oligo eliminated the signal from the GAPDH target (right-side image). Between each probe/quench oligo incubation and image recording, the blot was washed 4×5 min in 1×PBST, 5 mM EDTA.

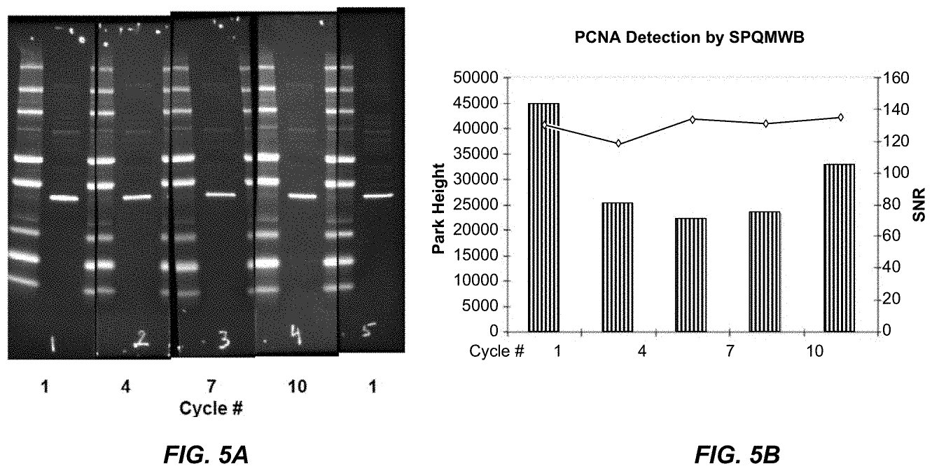

FIGS. 5A and 5B show that the signal-to-noise ratio for the Western blot detection of PCNA using the sequential multiplex method is stable over at least 10 probe-wash-detect-quench-wash cycles. Electrophoresis of 4-20% TGX gel containing alternating lanes of dual color standard and bug HEK293 lysate was performed, blotted to PVDF and cut into 5 strips, each with a lane of standards and a lane of lysate. The membrane strips were blocked for 30 min with 1×PBST (0.1% tween20), 3% BSA, followed by 1×PBST, 1% BSA, 5 mM EDTA, 100 ug/ml salmon sperm DNA and then placed into separate trays. Mouse anti-PCNA antibody-streptavidin conjugate (4 ug) was incubated 30 min with 200 pmol biotin capture oligo BC2 to form an antibody-oligo complex. The reaction was then diluted into 5 ml of blocking buffer and incubated at 4° C. overnight. The strips were washed 4×5 min with 1×PBST, 5 mM EDTA.

To mimic the sequential multiplex method, one strip was probed for 15 min with 50 nM BP2-cy5.5 oligo in 5 ml of Probe Buffer (1×PBST, 5 mM EDTA, 1% BSA, 5 ug/ml DNA), which anneals to the capture oligo associated with the PCNA antibody bound to the target on the blot. The remaining strips were mock-treated with the same buffer for the same time but without the probe oligo. After the probe step, the strips were again washed 4×, and then imaged using Chemidoc Touch (Bio-Rad) using the cy5.5 settings. The remaining strips were then incubated for another 15 min in the Probe Buffer followed by 4×5 min washes to mimic subsequent quench/probe-wash steps of the method. This process was repeated for a total of 10 cycles with detection of PCNA by the specific BP2-oligo being performed instead of mock detection at cycles 4, 7, and 10 using 3 of the other strips.

FIG. 5A shows detection at the same exposure time of PCNA using BP2-cy5.5 at cycles 1, 4, 7, and 10 and a repeat test at cycle 1. FIG. 5B shows a plot of signal (bars) and signal-to-noise ratio (line) across the different cycles.

In summary, the above example demonstrates the sequential detection of target proteins in a Western assay.

### Example 2

This Example describes different methods to reduce the signal from the detectable label.

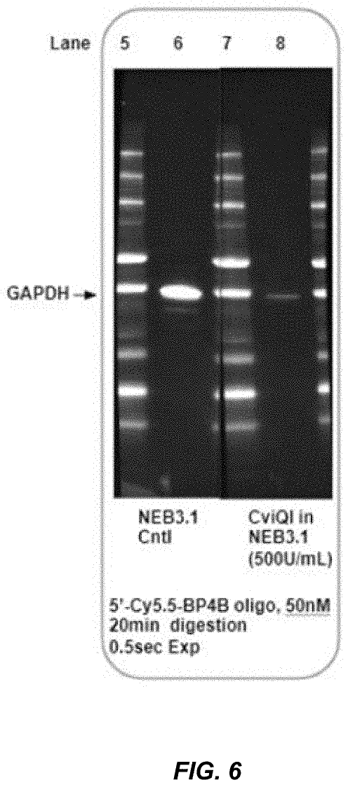

FIG. 6 shows representative results for a Sequential Multiplex Western Blotting assay where the signal from the target was gently removed at room temperature using a restriction enzyme (CviQI (G\hat{ }TAC, New England BioLabs # R0639). A pair of PVDF Western blot strips (control and test) containing Dual Color standard and HEK293 lysate (10 ug) was incubated overnight at 4 C with 5 ml of 0.8 ug/ml oligo-encoded anti-GAPDH-SA-BC4B antibody-oligo complex. After washing, the blots were then probed with 50 nM Cy5.5-labeled BP4B oligo in probe buffer for 15 min to detect the GAPDH (37 kDa) target. Subsequently, the membranes were incubated with either 1 ml of NEB3.1 buffer (Cntl, left-side image) or 1 ml of 500 U/ml of CviQI restriction enzyme (New England BioLabs # R0639) in NEB3.1 buffer (right-side image) for 20 min at room temperature, and then imaged for 0.5 sec on the cy5.5 channel of a ChemiDoc Touch (Bio-Rad Laboratories). The figure shows that the CviQI restriction enzyme effectively removes over 97% of the GAPDH fluorescent signal compared to a buffer control.

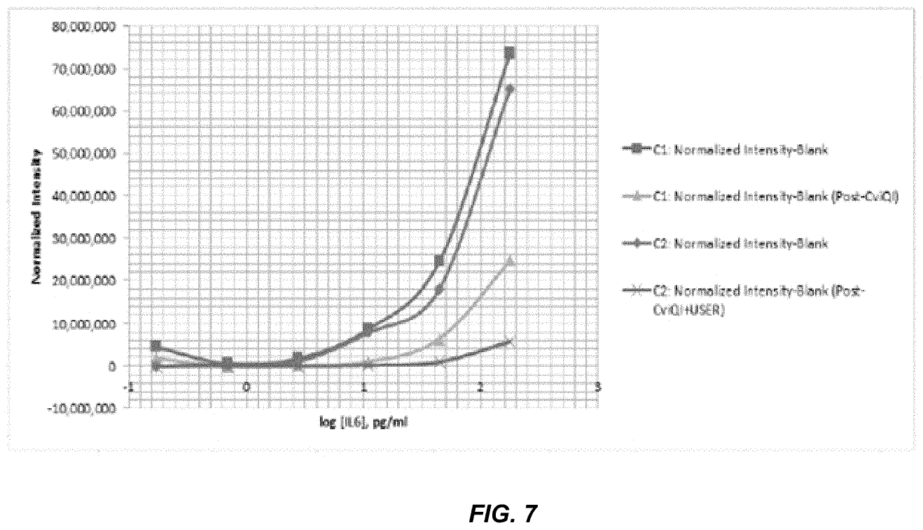

FIG. 7 shows that in some embodiments, the surface can be a magnetic particle and that the target signal can be removed or reduced using enzymes, such as a restriction enzyme alone or in combination with other enzymes such as USER (Uracil-Specific Excision Reagent, New England Biolabs). In one example, target IL-6 human antigen (0.17 to 177 pg/ml in standard diluent, Bio-Rad Labs, #171DK0001) was captured to magnetic particles containing an anti-IL6 antibody and then probed using a second human IL-6 specific mAb-streptavidin conjugate containing a unique oligonucleotide sequence (5′-biotin-BC4B) at 50 nM. The Ab-SA:oligo complex was prepared by incubating equal volumes of 1 mg/ml IL6-Ab-SA and 100 uM 5′biotin-BC4B oligo for 30 min at room temperature. The immune complex was detected by hybridizing a complementary oligonucleotide-5′-HRP conjugate (BP4D-HRP) and use of clarity max (Bio-Rad Labs) chemiluminescent substrate after focusing the beads using a magnet and imaging using ChemiDoc MP (Bio-Rad Labs). The target signal was then removed in a 15 min incubation at room temperature in 1×NEB3.1 Buffer using either CviQI (400 U/ml) restriction enzyme or CviQI (400 U/ml, triangle) plus USER (20 U/ml, X) and the chemiluminescent reaction repeated. As shown in FIG. 7, the enzyme mixture containing CviQI and USER removed the target signal to a greater extent than the restriction enzyme alone. There was still residual signal at the highest concentrations of target antigen, but this could be reduced or eliminated using further optimization. A control incubation with buffer (diamonds), did not result in loss of signal.

Preparation of IL-6 Capture Particles

Mouse anti-human IL-6 monoclonal capture antibody (Bio-Rad Labs, #1001228, lot 100002765, 3 ug) was conjugated to 6-8 um Absolute Mag carboxyl magnetic particles (Creative Diagnostics, # WHM-S034) using standard EDC/NHS chemistry. Briefly, 2.7e07 washed particles were first activated with 2.7 mg/ml sulfoNHS (ThermoFisher Scientific, PG82071) and 2.4 mg/ml EDC (ThermoFisher Scientific, PG82079) for 20 min at room temperature in 50 ul of 0.1M sodium phosphate pH 6.0 buffer. The particles were then washed in 0.1M MES buffer, pH 6.0 and resuspended with 50 ul of same buffer containing 3 ug of antibody. The coupling was performed for 1 hr at room temperature, after which, the beads were washed and then the surface blocked for 30 min using PBST buffer containing 1% BSA. The final bead preparation was stored at 4 C in TBST containing 0.02% sodium azide.

Preparation of IL-6 Streptavidin Conjugate

Anti-human IL-6 monoclonal detection antibody (Bio-Rad Labs, #1003064, lot 100003303) was conjugated to streptavidin using the Lynx Rapid Streptavidin Conjugation kit from Bio-Rad Laboratories (LNK161STR) as described above.

Preparation of BP4D-HRP Conjugate

5′-dithiol oligonucleotide BP4D was conjugated to EZ-link maleimide activated horseradish peroxidase (HRP) (ThermoFisher Scientific, #31485) in 0.1M sodium phosphate, pH 7.2 buffer containing 5 mM EDTA. Briefly, the 5′ dithiol oligonucleotide (IDT) was reduced with 80 mM DTT at 70 C for 5 min and then buffer-exchanged using Pierce 3K filter spin units (ThermoFisher Scientific, #88512) in the same buffer. Reduced oligo and maleimide-activated HRP, both in the same buffer were combined in approximately a 3:1 oligo:mal-HRP ratio and incubated for 2 hr at room temperature to react. The extent of conjugation was confirmed by Experion electrophoresis (Bio-Rad Labs), the solution was made to 50% glycerol and stored at −20 C. The conjugate was used without further purification.

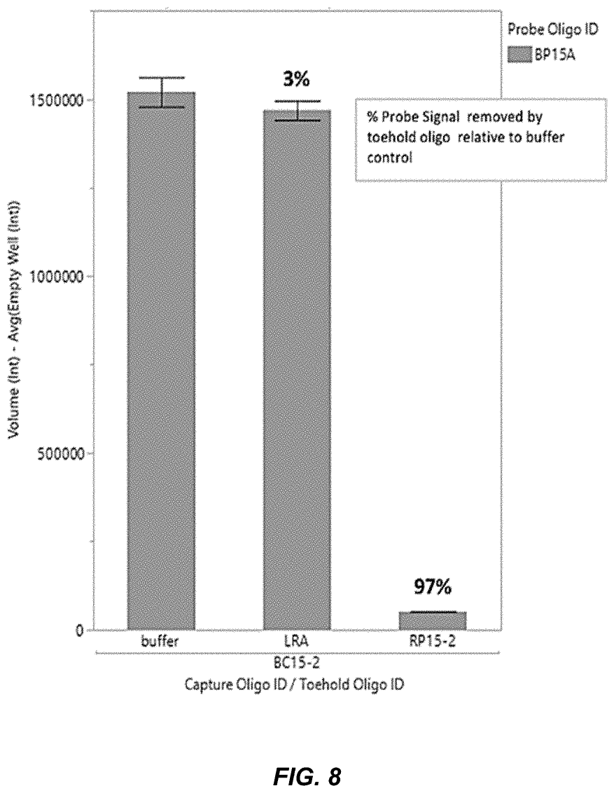

FIG. 8 shows that in some embodiments, the surface can be a magnetic particle and that target signal can be removed using a toehold exchange strand displacement oligonucleotide probe. The data shows that cy5.5 fluorescent signal from the Streptavidin:biotin-oligo cy5.5-labeled duplex formed on 1 um Dynabeads could be effectively removed using the complementary toehold probe that first anneals to the probe oligo strand through a 7 base single stranded region, while an off-target non-complementary control oligo and the buffer control failed to disrupt the labeled duplex.

Preparation of Streptavidin:BC15-2:BP15A Cy5.5-Labeled SA:Oligo Duplex Attached to Beads

In a tube, 30 ul (120 pmol biotin binding sites) of 1 mg/ml Streptavidin coated 1 um Dynabeads (ThermoFisher Scientific, #65601) were washed with 4× with 0.5 ml 1×PBS, 5 mM EDTA buffer, concentrating the beads each wash using a magnetic manifold. The beads were resuspended with 75 ul of buffer and 1.5 ul of 100 uM BC15-2 5′-biotin capture oligo were added to the SA-beads and incubated at RT for 15 min while shaking at 1000 rpm to form a SA bead:BC15-2 oligo complex. The beads were then washed 4× with 1×PBS, 5 mM EDTA, 10 ug/ml sheared salmon sperm DNA (PE5D10 buffer) and resuspended with 75 ul buffer. Next, 5′-cy5.5 labeled BP15A oligo was added to final concentration of 2 uM and incubated with shaking for 15 min at room temperature. The beads were again washed 4× with PE5D10 buffer and then resuspended with 700 ul of buffer.

Toehold Strand Displacement Assay

To assay for the toehold strand displacement as a means to remove signal, 50 ul of Cy5.5-labeled SA:oligo duplex beads were pelleted and then resuspended with either 50 ul of PE5D10 buffer (Control), 50 ul of 50 uM of an off-target toehold oligo (negative control), or 50 ul of 50 uM RP15-2 complementary toehold oligo, and incubated for 60 min at 1000 rpm at room temperature. At the end of the incubation, the beads were washed 3× with PE5D10 buffer and resuspended in 50 ul of same buffer. Aliquots of the reactions (2 ul) were spotted in triplicate and the beads concentrated using a magnetic manifold before imaging using a ChemiDoc MP imager (Bio-Rad Labs) set on the DyeLight680 channel. The volume intensities of each spot were determined and compared.

In summary, the above example demonstrates that the signal from the detectable label was reduced on a Western blot or magnetic particle.

All patents, patent applications, sequence accession numbers (e.g., Genbank accession numbers) and other published reference materials cited in this specification are hereby incorporated herein by reference in their entirety.

What is claimed is:

1. A method for sequentially detecting the presence of target analytes in a sample, comprising:
i) contacting the sample comprising two or more analytes immobilized on a solid support with two or more binding agents that specifically bind an analyte in the sample, wherein each of the analyte-specific binding agents binds a different analyte and is attached to a single-stranded nucleic acid molecule comprising a unique sequence;
ii) contacting the sample with a nucleic acid molecule comprising a first detectable label and a sequence having a region of complementarity that binds the unique sequence attached to a first analyte-specific binding agent;
iii) detecting a signal from a first detectable label;
iv) reducing the signal of the first detectable label;
v) contacting the sample with a nucleic acid molecule comprising a second detectable label and a sequence having a region of complementarity that binds a unique sequence attached to a second analyte-specific binding agent; and
vi) detecting a signal from a second detectable label,
thereby sequentially detecting the presence of the two or more analytes.

2. The method of claim 1, further comprising repeating steps (iv)-(vi) for each additional target analyte immobilized on the solid support.

3. The method of claim 1, wherein step (iv) and step (v) occur simultaneously.

4. The method of claim 1, wherein reducing the signal of the detectable label comprises quenching, inactivating, or removing the signal or detectable label.

5. The method of claim 4, wherein the removing comprises digesting the nucleic acid comprising the detectable label.

6. The method of claim 4, wherein the removing comprises strand displacement using a toehold probe or polymerase activity.

7. The method of claim 4, wherein reducing the signal of the detectable label does not remove target analytes from the solid support.

8. The method of claim 1, wherein the first and second detectable label is the same or different.

9. The method of claim 1, wherein the nucleic acid molecule comprising the detectable label forms a duplex along at least a portion of the unique sequence.

10. The method of claim 1, wherein the single-stranded nucleic acid molecule is attached to the binding agent via a 5′ phosphate group, an amine group, carboxyl group, hydroxyl group, a sulfhydryl group, click chemistry, copper(I)-catalyzed azide-alkyne cycloaddition (CuAAC), strain-promoted azide-alkyne cycloaddition (SPAAC), strain-promoted alkyne-nitrone cycloaddition (SPANC), or a linker.

11. The method of claim 10, wherein the linker comprises biotin, streptavidin, protein A, protein G, protein A/G, or protein L.

12. The method of claim 1, wherein the binding agent comprises an antibody or antigen-binding fragment thereof, an aptamer, a receptor, a ligand, a peptide, or a small molecule.

13. A method for sequentially detecting the presence of two or more target analytes in a sample, comprising:
i) contacting a solid support comprising at least two different target analytes immobilized thereon with at least a first binding agent that specifically binds a first target analyte in the sample and at least a second binding agent that specifically binds a second target analyte in the sample, wherein the first analyte-specific binding agent is attached to a first nucleic acid molecule comprising a unique sequence, and the second analyte-specific binding agent is attached to a second nucleic acid molecule comprising a unique sequence;
ii) contacting the first nucleic acid molecule with a nucleic acid molecule comprising a first detectable label and a sequence that binds the first nucleic acid molecule;
iii) detecting a signal from the first detectable label;
iv) reducing the signal of the first detectable label;
v) contacting the second nucleic acid molecule with a nucleic acid molecule comprising a second detectable label and a sequence that binds the second nucleic acid molecule; and
vi) detecting a signal from the second detectable label;
thereby sequentially detecting different target analytes in the sample.

14. The method of claim 13, wherein the first and second nucleic acid molecules are single stranded.

15. The method of claim 13, wherein the sequence that binds the first nucleic acid molecule is complementary to a region of the unique sequence of the first nucleic acid molecule, and the sequence that binds the second nucleic acid molecule is complementary to a region of the unique sequence of the second nucleic acid molecule.

16. The method of claim 13, wherein the nucleic acid molecule comprising the first detectable label forms a duplex along at least a portion of the first nucleic acid molecule, and the nucleic acid molecule comprising the second detectable label forms a duplex along at least a portion the second nucleic acid molecule.

17. The method of claim 13, wherein the first and second detectable labels are the same or different.

18. The method of claim 13, further comprising repeating steps (iv)-(vi) for each additional target analyte immobilized on the solid support.

19. The method of claim 13, wherein reducing the signal of the detectable label comprises quenching, inactivating, or removing the signal or detectable label.

20. The method of claim 19, wherein the removing comprises digesting the nucleic acid comprising the detectable label.

21. The method of claim 19, wherein the removing comprises strand displacement using a toehold probe or polymerase activity.

22. The method of claim 13, wherein the nucleic acid molecule comprising the detectable label forms a duplex along at least a portion of the first nucleic acid molecule, the second nucleic acid molecule, or both.

23. The method of claim 13, wherein the first nucleic acid molecule, the second nucleic acid molecule, or both are attached to the binding agent via a 5′ phosphate group, an amine group, carboxyl group, hydroxyl group, a sulfhydryl group, click chemistry, copper(I)-catalyzed azide-alkyne cycloaddition (CuAAC), strain-promoted azide-alkyne cycloaddition (SPAAC), strain-promoted alkyne-nitrone cycloaddition (SPANC), or a linker.

24. The method of claim 23, wherein the linker comprises biotin, protein A, protein G, protein A/G, or protein L.

25. The method of claim 13, wherein the binding agent comprises an antibody or fragment thereof, an aptamer, a receptor, a ligand, a peptide, or a small molecule.

26. The method of claim 13, further comprising repeating steps (iv)-(vi) with additional binding agents that bind different target analytes in the sample.

27. The method of claim 13, wherein the binding agent comprises an antibody or fragment thereof, an aptamer, a ligand, a peptide, or a small molecule.

28. A composition comprising one or more binding agents attached to one or more target analytes immobilized on a solid support, wherein the binding agent is conjugated to an nucleic acid molecule comprising a unique sequence.

29. The composition of claim 28, wherein the nucleic acid molecule comprises a duplex along at least a portion of the nucleic acid molecule.

30. The composition of claim 29, wherein the nucleic acid molecule comprises a first oligonucleotide attached to the binding agent and a second oligonucleotide comprising a detectable label hybridized to the first oligonucleotide.

31. The composition of claim 30, wherein the first oligonucleotide is attached to the binding agent via a 5′ phosphate group, an amine group, carboxyl group, hydroxyl group, a sulfhydryl group, click chemistry, copper(I)-catalyzed azide-alkyne cycloaddition (CuAAC), strain-promoted azide-alkyne cycloaddition (SPAAC), strain-promoted alkyne-nitrone cycloaddition (SPANC), or a linker.

32. The composition of claim 31, wherein the linker comprises biotin, protein A, protein G, protein A/G, or protein L.

33. The composition of claim 28, wherein the binding agent comprises an antibody or fragment thereof, an aptamer, a receptor, a ligand, a peptide, or a small molecule.

34. A method for producing the composition of claim 28, comprising:
contacting the binding agent to the target analyte immobilized on the solid support.

35. A kit comprising the composition of claims 28-33.
